(19) United States
(12) Patent Application Publication
(10) Pub. No.: US 2011/0071560 A1
(43) Pub. Date: Mar. 24, 2011

(54) Ear candle with safety insert

(76) Inventor: Russell Lee Sheppard, Auburn, CA (US)

(21) Appl. No.: 12/586,362

(22) Filed: Sep. 22, 2009

(51) Int. Cl. A61F 11/00; C11C 5/00

(52) U.S. Cl. 606/162; 431/292

(57) ABSTRACT

An ear candle made of wax impregnated muslin cloth in the shape of a cone to which a non-flammable filter insert is placed in the tip of the candle to catch any debris that may fall down the interior of the candle, while the candle is lit, and the tip is disposed in a human ear.

### BACKGROUND

This invention relates to “Ear Candles”, which are made by various manufacturers in a wide variety of forms, with different manufacturing techniques. The purpose of the ear candle is to funnel smoke into the ear with the use of fire to aid the body in releasing excess cerumen from the outer ear canal. Only smoke, not liquid wax, or other debris should enter the outer ear canal from the ear candling process.

### BRIEF SUMMARY OF THE INVENTION

The ear candle is a hollow candle constructed of muslin cotton in a hollow sphere designed to aid in the natural excretion of ear wax. The Ear Candle safety filter tip is a non-flammable filter placed in the tip of a an ear candle to catch any debris that may fall down the candle. This safety filter insert can and should be used in any and all ear candles. The stop burn line is a line on an ear candle created to show a user the suggested location along the length of the ear candle where the user should extinguish the flame.

### BRIEF DESCRIPTION OF THE FIGURES

FIG. 1 is a perspective view of an ear candle having this invention.

FIG. 2 is an elongated sectional view of the ear candle of this invention.

FIG. 3 is a closeup sectional view of the tip of the ear candle of this invention.

FIG. 4 is a diagrammatic view to illustrate the known mode of utility of the class of devices of this invention.

### DESCRIPTION OF THE PREFERRED EMBODIMENT

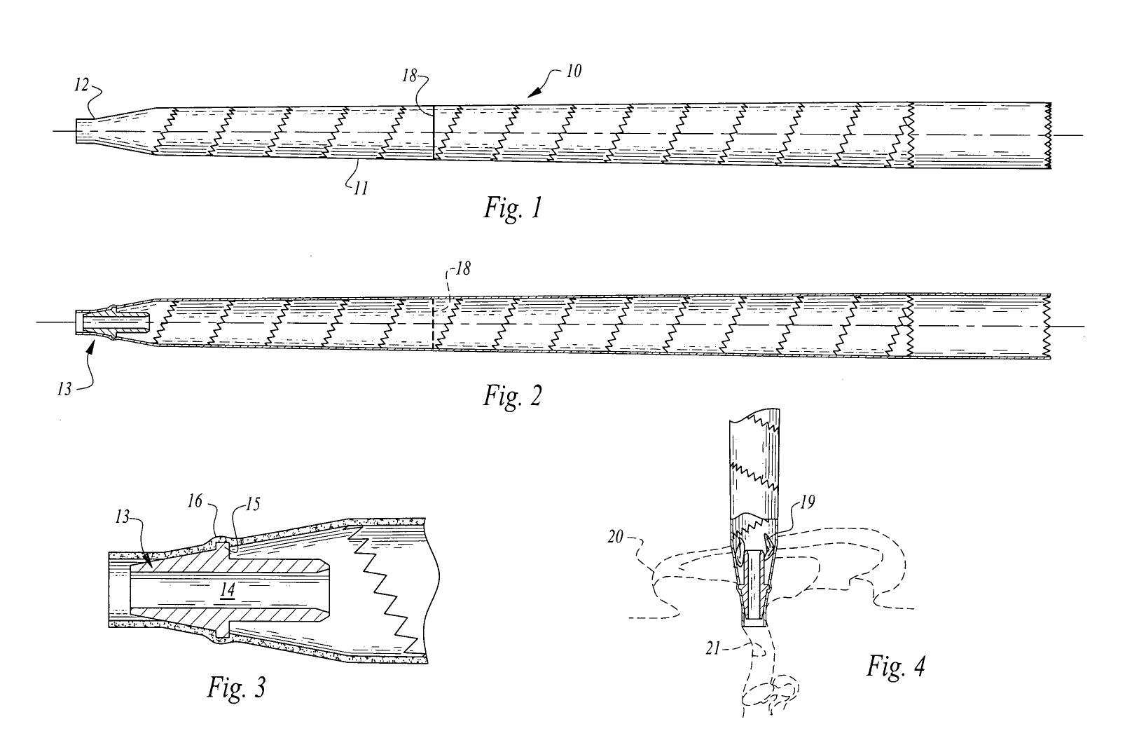

FIG. 1 shows the outside of the candle. The ear candle is made with a muslin cotton strip which has a pinking cut on the edge. This strip is formed into a cylindrical shape to create a hollow candle 10.75 inches long. The cylinder candle narrows into a tip as shown in, FIG. 2, 0.92 inches from the end of the tip end of the candle. The tip narrows to an opening that is 0.124 inches wide, designed to allow smoke to go through. Ear candles range from 5 to 15 inches with diameters from 0.25 to 1 inch. FIG. 3 shows the inside of the candle mostly focused on the Safety Filter Tip. This Safety Filter Tip was designed to catch any debris or wax due to misuse of the user. The Safety Filter insert is 0.75 inches long with a hole down the center which is 0.124 inches wide to match the hole of the ear candle to allow smoke to go through. The top half of the Safety Filter insert is designed with a lip around the cylinder section to create a ledge for any debris on the inside of the candle to land on. This pocket or ledge 15 is designed to catch any debris coming down the candle during use without interrupting the smoke flow. This filter tip is made from HIPS (High-Impact Polystyrene) but the invention is not specific to any specific material. FIG. 3 shows the candle cut in half. it is a hollow cylinder, with only the above mentioned filter insert inserted inside. The “Stop Burning” line is painted four inches from this tip. This distance from the tip of the ear candle to ensure maximum safety when one uses the ear candle.

An Ear Candle as is known in the homeopathic healing arts, is comprised of muslin cotton strips with a pinking shears cut edge on both sides, which strips are embedded with wax. A Safety filter insert is inserted into the narrow tip of the hollow candle to prevent debris from falling through the hole in the candle tip, thus allowing only smoke to blow through.

The candle of this invention, is used to help excrete ear wax from a person's ear. With the patient lying on his/her side with the ear to be treated facing upwardly, the burning candle's tip is inserted into the inner ear such that the smoke from the burning candle drift down through the bore 14 of the insert 13 can enter to soften the wax. After a finite time, the candle is removed, the head is turned 180 degrees and the wax is permitted to flow out downwardly from the treated ear onto a pillow or other surface. The previously noted stop burn line, 18, is a protection measure to give the user a visual indication of where on the length of the candle to stop the flame and extinguish the candle.

The Safety Filter insert is placed into the narrow tip of the candle at the time of manufacture of the candle. See FIG. 3. This insert is made from a non flammable plastic and is designed to direct any debris on the interior of the candle—melting candle wax—as can be seen in FIG. 4, into the side 15 of the safety filter insert 13 between the narrow hole and the muslin candle at location 16. There is a passageway or bore 14 down the center of the insert 13 to allow for smoke to go through. The insert is also used to strengthen and support the candle and allow the ear candle to keep the integrity of its shape and design.

All matter recited herein is to be considered as exemplary and should not be considered as limiting.

1. An ear candle made out of an elongated strip of muslin cotton with a pinking cut edge, embedded with wax, and in the shape of a cylinder cone tapered from the wide open first end to a narrower second end, and having a safety insert which is intended for insertion into the outer ear canal disposed therein, said safety insert being adapted to retain melted wax from the candle, when said candle is burning, from entering the ear canal.

2. The ear candle of claim 1 wherein said candle has a stop burn line painted onto it four inches from the tip of the ear candle.

3. The ear candle of claim 1 wherein said candle has inserted therein a filter tip molded from a high-impact polystyrene.

4. (canceled)

5. The Safety Filter insert of claim 6 wherein said insert allows smoke to channel through its center hole and to stop candle debris from falling through the hole by directing the debris to an outer chamber that is created between the Safety Filter insert and the ear candle wall onto the circumscribing lip.

6. A Safety Filter insert for disposition within an ear candle tip during the course of manufacture, having an elongated central bore within a body having a tapered lower end, an intermediate circumscribing lip along the length thereof extending outwardly beyond the edge of the taper said insert also having a tubular upper end.

7. The safety filter insert of claim 6 wherein said insert is molded from high impact plastic.

8. The insert of claim 7 wherein the plastic is high impact polystyrene.
